(19) United States
(12) Patent Application Publication
(10) Pub. No.: US 2017/0216024 A1
(43) Pub. Date: Aug. 3, 2017

(54) SEALING ELEMENTS FOR INTRACARDIAC DEVICES

(71) Applicant: MVALVE TECHNOLOGIES LTD., Herzliya (IL)

(72) Inventors: Shay DUBI, Tel Aviv (IL); Amit TUBISHEVITZ, Tel Aviv (IL)

(21) Appl. No.: 15/501,023

(22) PCT Filed: Jul. 30, 2015
(86) PCT No.: PCT/IL2015/050786

§ 371 Date: Feb. 1, 2017

Related U.S. Application Data
(60) Provisional application No. 62/032,581, filed on Aug. 3, 2014.

(51) Int. Cl. A61F 2/24

(52) U.S. Cl. CPC A61F 2/2409; A61F 2/246; A61F 2220/0016; A61F 2250/006; A61F 2250/0069

(57) ABSTRACT

The present invention provides an intracardiac device (60) suitable for endovascular and/or transapical implantation at a cardiac valve annulus, wherein said device comprises means for reducing paravalvular leakage (PVL), said means being selected from the group consisting of: lateral-edge extensions, one or more tubular sealing elements, one or more barbs, an inferiorly-directed circumferential fabric skirt (68) attached to the inner circumference of the intracardiac device and an inferiorly-directed fabric curtain attached to the outer circumference of said device.

### FIELD OF THE INVENTION

The present invention relates to intracardiac devices that incorporate means for reducing paravalvular leakage. In particular, the present invention provides valve support devices with improved annular sealing properties.

### BACKGROUND OF THE INVENTION

Paravalvular leakage (PVL) is a well-known complication associated with the implantation of prosthetic intracardiac devices, such as replacement valves. Depending on the severity of the leakage, the consequences of PVL can range from the clinically insignificant to severe and even fatal outcomes, such as congestive heart failure. Thus, it has been observed by the present inventors that following the lateral displacement of the native mitral valve leaflets that occurs during the implantation of a replacement heart valve within a previously-deployed mitral valve support device (as disclosed, for example, in co-owned, co-pending international patent applications PCT/IL2013/000025, published as WO 2013/128436, and PCT/IL2013/000036, published as WO 2013/150512), leakage may occur between the lateral perimeter of said valve support device and the atrial wall.

Clearly, it is essential to overcome this problem of leakage associated with intracardiac devices, in order to prevent the adverse clinical effects mentioned hereinabove. The present invention provides several different strategies for preventing and overcoming this problem.

### SUMMARY OF THE INVENTION

The present invention is directed to an intracardiac device suitable for endovascular and/or transapical implantation at or near to a cardiac valve annulus, wherein said device comprises means for reducing PVL.

In one highly preferred embodiment, the intracardiac device is an annular, or ring-shaped, device characterized in having an inner circumference and an outer circumference. In one particularly preferred embodiment the ring-shaped intracardiac device is a valve support device intended for use in conjunction with a replacement cardiac valve. As mentioned hereinabove, the intracardiac device of the present invention is suitable for delivery and implantation by the endovascular and/or transapical routes. In this context, the term “is suitable for” refers to the fact that the device is capable of being folded or collapsed (“crimped”) into a low profile, small-diameter conformation that will enable its delivery through a similarly small-diameter delivery catheter or other device. Then, after haven being delivered to the desired implantation site (e.g. the mitral valve annulus), the intracardiac device is released from the confines of the delivery device and allowed to expand into its working conformation. This process of crimping and expansion is, in most embodiments, facilitated by the fact that the intracardiac device is constructed of a shape-memory material such as Nitinol.

In the context of the present invention, the term “valve support device” is used to refer to any intracardiac device that is adapted for implantation at a cardiac valve annulus (such as—but not limited to—the mitral annulus). The purpose of such devices is to provide a stable platform for the implantation and deployment of a prosthetic valve at said cardiac valve annulus. While the prosthetic valve may deployed at the same time as valve support device, in many cases (for example, as described in the co-owned, co-pending patent application that published as US 2014/0005778), the prosthetic valve is deployed following implantation of the valve support device, as the second stage in a two-stage procedure.

One of the key advantages of using a valve support device as a platform (rather than directly deploying a prosthetic valve at the annulus) is that this permits the use of standard, commercially-available prosthetic valves, since the stabilization and anchoring within the cardiac tissues becomes a function of the specialized valve support device—rather than necessitating the modification of the prosthetic valve in order to incorporate stabilizing elements. Furthermore, in the case of mitral valve replacement, the valve support device also serves to effectively reduce the diameter of the large mitral valve annulus, thereby facilitating the deployment of a smaller diameter aortic valve within the central space of said support device. In this regard it should be noted that in many (but not all) cases, the valve support device has an annular (or ring-shaped) form, the outer circumference of which engages with the cardiac tissues, while the inner circumference of the “ring” defines a central space into which a prosthetic heart valve may be stably implanted.

In some cases, the cardiac valve support device may comprise a single support ring, as disclosed in co-owned, co-pending WO 2013/128436, the contents of which are incorporated herein in their entirety. In other cases, the support device may comprise two rings mutually connected by bridging elements, as disclosed in co-owned, co-pending US 2014/0005778, the contents of which are incorporated herein in their entirety. However, it is to be recognized that the sealing elements of the present invention are also intended for use in conjunction with other forms of cardiac support device not disclosed in these two publications.

In one particularly preferred embodiment the intracardiac device is a cardiac valve support device comprising a single support ring (as disclosed in co-owned WO 2013/128436). Generally, such a valve-support device comprising a single ring-shaped annular support element, has a collapsed delivery configuration and a deployed configuration. In one embodiment, the support element is provided in the form of flat annular ring, preferably constructed from a material having superelastic and/or shape memory properties. One example of such a suitable material is Nitinol, which possesses both of the aforementioned physical properties. These properties may be utilized in order to permit said device, following its delivery in a collapsed conformation, to return to an expanded memory configuration after being heated above its transition temperature. In the radial plane (i.e. the plane in which the native cardiac valve leaflets are disposed when in their closed position), the size of the annular support element may be defined in terms of its outer radius (Ro), its inner radius (Ri) and the difference between these two radii (Rd). It should be appreciated that Ro is determined by the diameter of the mitral valve annulus into which the valve support device will be implanted. Ri, however, is determined by the outer diameter of the replacement heart valve that will be inserted into the central space of the support device. Generally, the prosthetic aortic valves used in conjunction with the valve support device of the present invention have an external diameter considerably less than that of the mitral valve annulus. It may therefore be appreciated that Rd approximately corresponds to the annular gap between the small outside-diameter replacement valve and the relatively large diameter mitral valve annulus. Preferably, Rd is in the range of 1-14 mm. In most embodiments of the valve support device of the present invention, the inner radius of the single-ring support element is in the range of 23-29 mm and the outer radius thereof is in the range of 30-50 mm.

The above-described single-ring valve support device is particularly suitable for two-step endovascular and/or transapical implantation procedures for replacing a patient's native mitral valve. In general, the support structure is first delivered in a collapsed conformation within a delivery device and positioned near or within a mitral valve annulus and secured in place. A replacement mitral valve is subsequently secured to the support structure, securing the replacement valve in place near or within the annulus. By implanting the support structure and replacement mitral valve in two steps, the replacement mitral valve can have a lower delivery profile because it does not have to expand as much to contact native tissue due to the presence of the support structure. This eliminates the need to have a large delivery profile replacement valve as would be required if attempting to position an aortic valve in the native mitral valve, or if attempting to position a one-piece mitral valve implant (i.e., an implant not assembled in-vivo) within the native mitral valve. Examples of suitable delivery systems that may be used to implant the single-ring valve support device of the present invention are disclosed in co-owned WO 2013/128436 and WO 2014/128705, the contents of both of which are incorporated herein in their entirety.

In one preferred embodiment, said valve support device is suitable in size and shape for implantation at the mitral valve annulus. In another preferred embodiment, the valve support device is suitable in size and shape for implantation within or adjacent to the aortic valve. With regard to the size of valve support devices suitable for implantation at these two anatomical sites, support devices intended for use at the mitral valve annulus will generally have an external radius in the range of 25 to 55 mm, while support devices intended for use at the aortic valve annulus will generally have an external radius in the range of 20 to 35 mm.

In another preferred embodiment, the intracardiac device is a prosthetic cardiac valve of a generally tubular shape, having an annular or ring-shaped cross-sectional shape.

Preferably, the means for reducing PVL are selected from the group consisting of: lateral edge extensions, one or more tubular sealing elements, one or more barbs, an inferiorly-directed circumferential fabric skirt attached to the inner circumference of the annular intracardiac device and an inferiorly-directed fabric curtain attached to the outer circumference of said device.

It is to be noted that the present invention also encompasses the use of a combination of more than one of the above mentioned sealing means, or a combination of one of the means with partial use of another means. For example, particularly preferred embodiments include a device with both a tubular sealing element and a fabric skirt, or a tubular sealing element and a partial fabric skirt covering only part of the circumference of the device.

### BRIEF DESCRIPTION OF THE DRAWINGS

FIG. 1 depicts a valve support device that is suitable for implantation at the mitral position, in which the position and direction of the undesirable paravalvular flow is indicated by arrows.

FIG. 2 diagrammatical represents a valve support device fitted with lateral edge extensions which function as means for reducing PVL.

FIG. 3 is a photographic representation of a mitral valve support device held within a delivery system, wherein said valve support device comprises a tubular sealing element.

FIG. 4 depicts, in plan view, a valve support device in cut-out form, prior to being crimped within a delivery system, wherein said support device comprises barb-like means that enable improved anchoring of said device within the cardiac tissue, thereby reducing PVL.

FIG. 5 presents a side view of the valve support device shown in FIG. 4, in which the barb-like structures are in their deployed configuration.

FIG. 6 shows a valve support device comprising a full-circumference skirt attached to the inner perimeter of the support ring.

FIG. 7 provides a view of the inferior surface and lateral aspect of a valve support ring having a sealing curtain attached to the outer circumference of the support ring.

The various embodiments of the present invention will now be described with reference to the above-listed drawings.

### DETAILED DESCRIPTION OF THE PREFERRED EMBODIMENTS

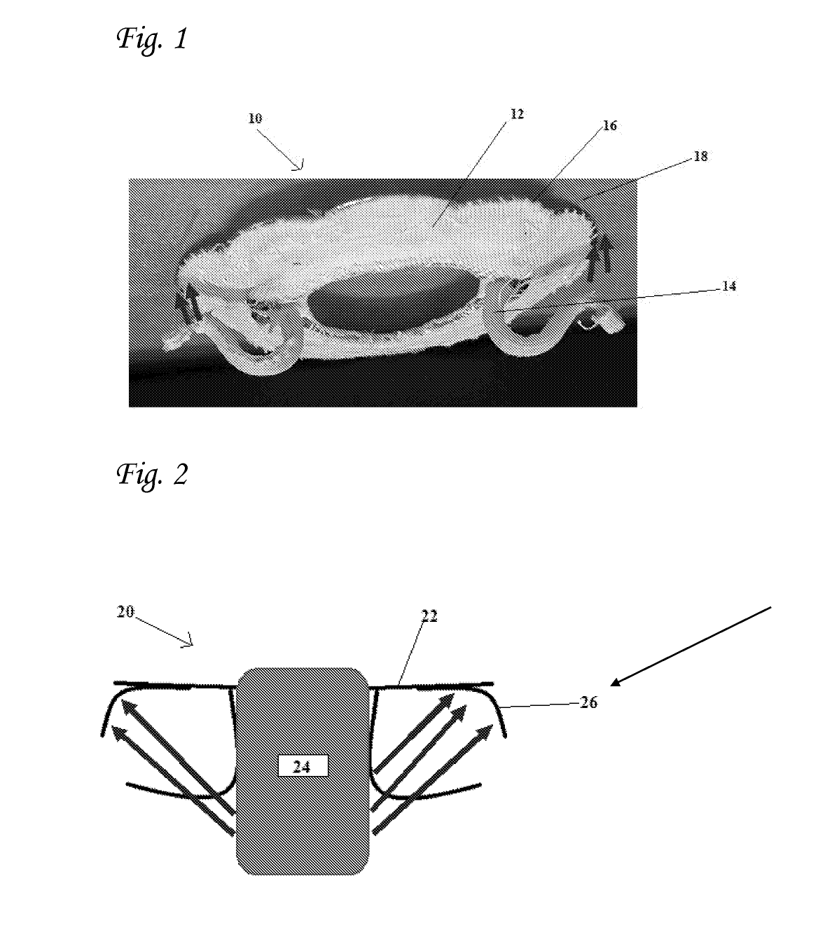

As explained hereinabove, significant PVL can occur between an intracardiac device implanted within a valve annulus and the adjacent cardiac tissues. This state of affairs is shown in FIG. 1 which depicts a mitral valve support device 10 comprising a single support ring 12 and two stabilizing wings 14. Curved lines 18 are intended to show the position of the atrial wall in relation to support device 10, after said device has been implanted at the mitral annulus. The pairs of upwardly-pointing arrows indicate the position and direction of the PVL, that is, into the pocket-like region where the outer circumference of the annular support device meets the atrial wall. In many cases, the leakage problem is exacerbated by the presence of a replacement valve within the central space of the annular valve support device, said valve causing the lateral displacement of the native valve leaflets and altering the topology of the region of the annulus adjacent to the implanted support ring.

The present inventors have found that it is possible to overcome this leakage problem by means of altering the shape of the lateral portion of the valve support ring. In one embodiment, this may be achieved by the presence of an additional lateral extension having an origin on the support ring and a free edge that extends latero-inferiorly from said origin. In another embodiment, the outer portion of the support ring itself is caused (during manufacture) to curve laterally and inferiorly. In either case, the lateral extension or the downwardly curved outer ring portion is constructed such that it is very flexible, thereby enabling it to conform to the anatomy of the atrial wall. In one preferred embodiment, the lateral extension or curved outer ring portion is constructed from Nitinol having a thickness of 0.1-0.5 mm, preferably 0.1 or 0.2 mm.

FIG. 2 illustrates the first of the two above-mentioned embodiments, in which valve support device 20 comprises a single support ring 22 and a lateral extension 26 which extends around the entire circumference of said ring. Replacement valve 24 has been implanted within the central space of said support device. It will further be seen in this figure that the flow of blood (indicated by the diagonally-orientated arrows) in the region of said replacement valve ends at the lateral extension, which thereby prevents the leakage of blood around the lateral edge of support ring 22. Furthermore, the fluid flow itself at this point will tend to cause a small displacement of the lateral extension in an upward and lateral direction, thereby further improving the fluid seal provided by said extension.

A further solution found by the present inventors is the use of a sealing ring attached to the outer portion of the annular intracardiac device, wherein said ring may be either continuous around the entire circumference of said device (i.e. similar to the form of an O-ring) or may be discontinuous, consisting of discrete portions.

In one preferred embodiment, the sealing ring may have different elasticity values in different location. Thus, for example, the ring may have a higher elasticity in the area of the aorta.

In one preferred embodiment, said sealing ring may be constructed from a braided tube (for example made of Nitinol wires, or wires constructed from another biocompatible metal) covered with a biocompatible fabric (such as PTFE, Dacron, Polyester or other biocompatible fabrics). The braided tube may be manufactured in two steps, wherein the first step comprises braiding the said wires into a tube shape, and the second stage consists of covering the braid with biocompatible fabric. The braid may be formed into a ring shape by closing the free edges thereof. Exemplary dimensions for such braided tubes are wires having a thickness of 0.03 mm-1 mm, exemplary number of wires in the braiding may be 8-64 wires, and typically the diameter of the braided tube may be 2-15 mm.

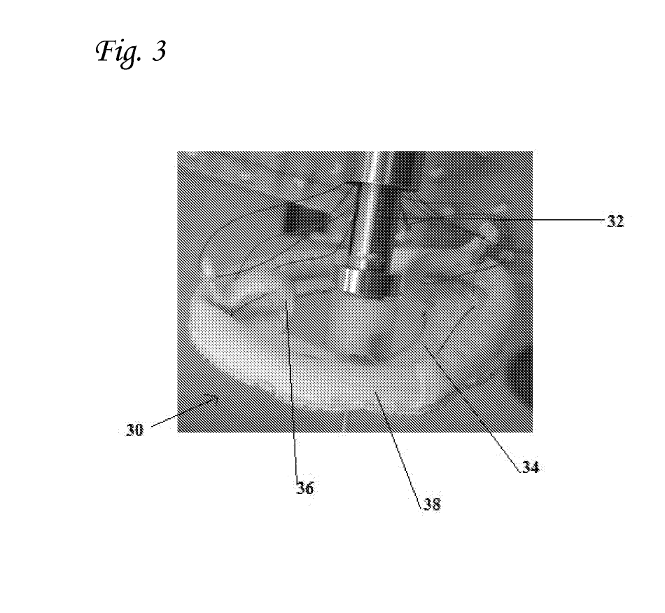

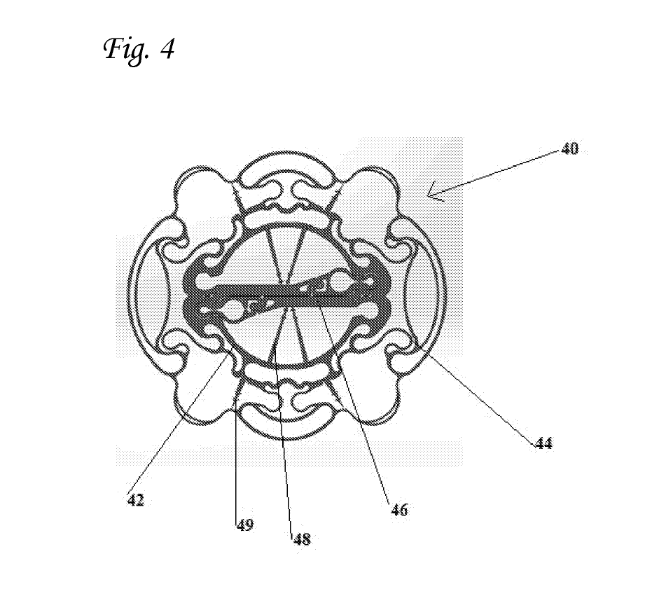

One example of this embodiment is illustrated in FIG. 3 which shows a mitral valve support device 30 comprising a single support ring 34 and two stabilizing or anchoring wings 36 held within a delivery device 32 by a series of wires. As shown in this figure, a complete sealing ring 38 constructed from a fabric-covered braided tube is attached to the outer portion of said support ring. In this example, the wire used to manufacture the braided tube has a diameter of 0.1 mm while the tube itself has an external diameter of 4.6 mm.

During use, the sealing ring will become compressed against the cardiac tissues of the atrial wall and mitral annulus, thereby ensuring complete sealing at all stages of the cardiac cycle, and thus preventing PVL.

A unique feature of the braided tube of the invention is the fact that its mechanical characteristics allow it to apply forces on the cardiac tissue when the device is deployed in its working position (said forces rising from the expansion of the braid), and thus the fabric of the braid (which does not allow blood penetration) is approximated to the cardiac tissue in different anatomic positions, different anatomic sizes and shapes, and in different parts of the cardiac cycle—maintaining a constant sealing to prevent leakage. At the same time, and very importantly, the braided structure allows crimping of the intracardiac device to very small size, thereby enabling transcatheter delivery of the device. By way of example, a braided sealing tube which in its “resting” state has a diameter of approximately 7 mm, braided from 42 wires which are 0.06 mm thick, can be crimped to a diameter of less 1 mm.

In another embodiment of this aspect of the invention, the sealing ring is constructed from a metallic sponge-like material (e.g. a metallic wool).

A further approach that has been adopted is to use barb-like prongs attached to various portions of the annular intracardiac device, wherein the free ends of said prongs become embedded within the cardiac tissue, thereby improving the apposition of said device to the cardiac tissues and thus preventing or reducing PVL. FIG. 4 illustrates one version of this embodiment, a mitral valve support device 40 comprising a single support ring 42 connected on its lateral surface to an intricate crown-like lateral portion 44 and two stabilizing wings 46. (Said valve support device is depicted in this figure in flat form, after having been cut out of a Nitinol sheet, but before being crimped into a delivery device.) It will be noted that this support device comprises a total of eight prongs fitted with barbs—four relatively long prongs 48 attached to the inner circumference of support ring 42, and four shorter prongs 49 attached to the crown-like lateral portion 44.

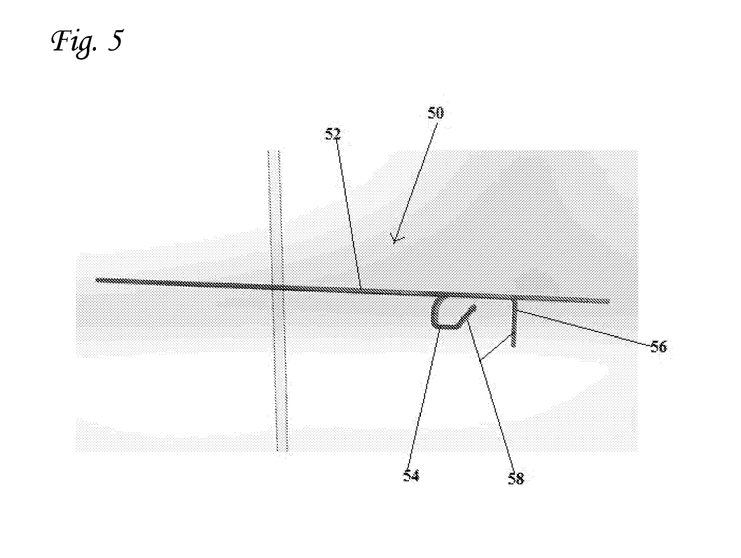

FIG. 5 illustrates a side view of the same embodiment as shown in FIG. 4, in its in situ conformation. It may be seen from this figure that the lateral prong 56 is straight and angled at about 90 degrees downwards (in relation to the plane of the support ring), while the medial prong 54 adopts a curved conformation. It should be noted that these two prong conformations are shown for illustrative purposes only, and various other shapes are also included within the scope of the invention.

In another aspect, the present invention also encompasses the use of a skirt-like fabric structure attached to the inner perimeter of the intracardiac device. In one preferred embodiment of this aspect, said skirt-like structure is attached to the entire inner circumference of said device and is disposed such that the body of said skirt passes inferiorly from said device. Said skirt can be made of a biocompatible fabric, for example PTFE, polyurethane, polyester and/or Dacron, and can be sutured to the device with a biocompatible surgical suture. The thickness of the fabric is preferably in the range of 0.05 mm to 1 mm. In certain embodiments, the skirt may be constructed from a biological material such as pericardium. In some embodiments the skirt may be constructed from two or more different fabrics and/or biological materials. In other preferred embodiments, the skirt may be constructed from materials (either a single material or a combination of materials) having at least two different thicknesses in different portions thereof. In certain cases, the variation in thickness is achieved by means of sewing one or more additional pieces of fabric onto a region of the fabric skirt. This additional piece may either be constructed of the same material as the skirt, or alternatively may comprise a different fabric. The thickness of the additional piece of fabric generally has a thickness within the range of 0.05 mm to 1 mm. The height of the additional fabric piece may be in the range of a few millimeters to the full height of the fabric skirt. Similarly, the width of each additional fabric piece may be in the range of a few millimeters to half the width of the fabric skirt. In one preferred embodiment, two additional pieces of fabric are attached to the skirt, separated such that when the skirt is attached to the intracardiac device, said pieces are separated by about 180 degrees around the circumference of said device. One advantage of this variable thickness embodiment is that in some cases, it can significantly improve the ability of the skirt to fully cover the subsequently-implanted prosthetic valve after said valve has been deployed within the central space of the valve support device. In particular, the presence of one or more additional pieces of fabric which have been sewn onto the skirt, has been found to significantly reduce the undesirable folding of said skirt during prosthetic valve deployment. In some cases, the desired shape of the fabric skirt will be created by means of constructing said skirt from a thermosetting fabric such as Polyester and applying a source of heat to said material. For example, the thermosetting fabric can be molded to the desired shape using a mandrel at a temperature of approximately 150 degrees Celsius for approximately 15 minutes and attached to the inner surface of the intracardiac device by means of surgical sutures.

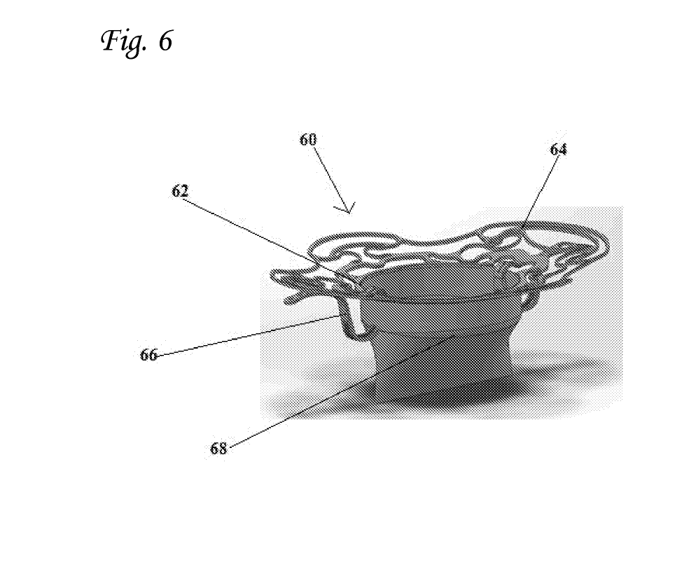

FIG. 6 illustrates one preferred embodiment of this aspect, in which a mitral valve support device 60 comprising a single support ring 62, a crown-like lateral extension 64 and two stabilizing wings 66 is fitted with a full-circumference fabric skirt 68. As shown in this figure, the lower portions of the fabric skirt may be mutually apposed, while the upper region has a circular outline at its attachment point on the support ring circumference. During the subsequent implantation of a prosthetic valve within the inner space of support ring 62, the apposed lower portions of skirt 68 will be separated and caused to form a generally tubular structure that covers the wall of the implanted valve. Since the prosthetic valve wall is entirely covered (on its lateral aspect) by the skirt-like structure of this embodiment, PVL associated with the presence of said prosthetic valve is significantly reduced or eliminated.

In yet another aspect, the leakage problem has been solved by the present inventors by means of fitting the intracardiac device with an inferiorly-disposed sealing drape attached to the outer circumference thereof. While the length of said drape (measured from its point of attachment on the intracardiac device to its lower free end) may have any suitable or desired value, in one preferred embodiment, said drape has a length of about 10-20 mm. In one preferred embodiment the drape is constructed from a biocompatible fabric, for example PTFE, polyurethane, polyester and/or Dacron and is attached to the intracardiac device by means of surgical sutures. In certain embodiments, the drape may be constructed from a biological material such as pericardium. In some embodiments the drape may be constructed from two or more different fabrics and/or biological materials. In other preferred embodiments, the drape may be constructed from materials (either a single material or a combination of materials) having at least two different thicknesses in different portions thereof.

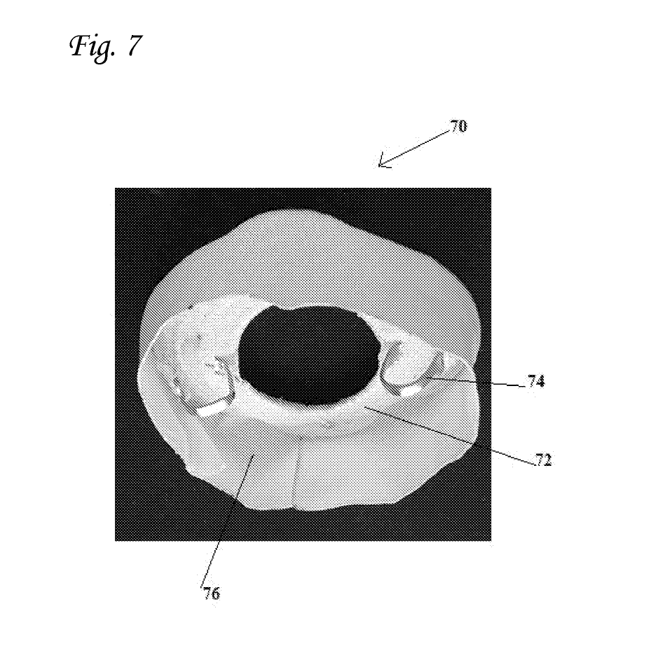

FIG. 7 illustrates one preferred embodiment of this aspect of the present invention, in which a mitral valve support device 70 comprising a single support ring 72 and two stabilizing wings 74 further comprises a fabric drape 76, wherein said drape is attached to the outer circumference of support ring 72. As will be noted from this figure, the drape is attached in a continuous manner to the outer edge of the support ring and then passed downwards (for about 20 mm, in the present example), such that when the device is implanted at the mitral annulus, the region that is most susceptible to PVL (i.e. the angle created between the mitral support ring and the atrial wall) is covered by said drape. In this way, PVL (and especially leakage associated with the presence of a subsequently-implanted prosthetic valve) is largely prevented.

In other embodiments of this invention the fabric drape may be attached to any other aspect of the ring, instead of, or in addition to the outer circumference.

In other embodiments instead of having one fully circular fabric drape, the seal may be made of two or more fabric drapes, overlapping one another and together forming a fully circular drape. The advantage of this structure is that this allows the device to crimp to a lower crimp diameter, which is important for transcatheter delivery.

The drape may also be a partial drape (not fully circular), for example a partial drape only in the area which will be approximated to the Anterior (aortic) mitral leaflet, and improve sealing in that area.

1. An intracardiac device suitable for endovascular and/or transapical implantation at a cardiac valve annulus, wherein said device comprises means for reducing paravalvular leakage (PVL), said means being selected from the group consisting of: lateral edge extensions, one or more tubular sealing elements, one or more barbs, an inferiorly-directed circumferential fabric skirt attached to the inner circumference of the intracardiac device and an inferiorly-directed fabric curtain attached to the outer circumference of said device.

2. The intracardiac device according to claim 1, wherein said device has an annular shape.

3. The intracardiac device according to claim 2, wherein said device is a valve support device.

4. The intracardiac device according to claim 3, wherein said valve support device is suitable in size and shape for implantation at the mitral valve annulus.

5. The intracardiac device according to claim 3, wherein said valve support device is suitable in size and shape for implantation at the aortic valve annulus.

6. The intracardiac device according to claim 1, wherein the means for reducing PVL comprise an inferiorly-directed circumferential fabric skirt attached to the inner circumference of said device.

7. The intracardiac device according to claim 6, wherein the fabric skirt has at least two different thicknesses in different portions thereof.

8. The intracardiac device according to claim 1, wherein the means for reducing PVL comprise an inferiorly-directed fabric curtain attached to the outer circumference of said device.
